(19) United States
(12) Patent Application Publication
(10) Pub. No.: US 2013/0331727 A1
(43) Pub. Date: Dec. 12, 2013

(54) METHOD AND SYSTEM FOR DETECTING ATTENTION

(71) Applicants: Haihong Zhang, Singapore (SG); Cuntai Guan, Singapore (SG); Chuanchu Wang, Singapore (SG)

(72) Inventors: Haihong Zhang, Singapore (SG); Cuntai Guan, Singapore (SG); Chuanchu Wang, Singapore (SG)

(73) Assignee: AGENCY FOR SCIENCE, TECHNOLOGY AND RESEARCH, Singapore (SG)

(21) Appl. No.: 13/982,204

(22) PCT Filed: Jan. 28, 2011
(86) PCT No.: PCT/SG2011/000042

§ 371 Date: Aug. 30, 2013

(51) Int. Cl. A61B 5/16; A61B 5/04

(52) U.S. Cl. CPC A61B 5/168; A61B 5/04012
USPC 600/544

(57) ABSTRACT

A method and system for detecting attention of a subject is provided. The method comprises determining one or more user specific feature sets from Electroencephalographic (EEG) signals of the subject; determining one or more pool data feature sets from the EEG signals of the subject; identifying one or more features from each feature set for differentiating attention/non-attention signals; determining respective classification scores for each feature set based on the identified one or more features; and combining the classification scores to obtain a combined attention score for said detecting attention.

### FIELD OF INVENTION

The present invention relates broadly to a method and system for detecting attention.

### BACKGROUND

Attention-deficit/hyperactivity disorder (ADHD) is a psychiatric condition and a neuro-developmental disorder with various hyperactivity, impulsivity and inattention symptoms. Stimulant medication is typically used for treatment but concerns over medication are increasing in terms of, for example, effect size, adverse effects as well as long-term efficacy. Therefore, alternative interventions for ADHD are desired.

An alternative treatment, Electroencephalographic (EEG) biofeedback (also known as neurofeedback), has been researched since the 1970s. Neurofeedback is an operant conditioning procedure in which a patient learns to self-control certain EEG patterns while guided by real-time visual or acoustic representations of the EEG patterns. This is discussed in D. J. Fox et. al., Neurofeedback: An alternative and efficacious treatment for attention deficit hyperactivity disorder, Applied Psychophysiology and Biofeedback, 30:365-373, 2005. Recent reviews can be found in, for example, L. Thompson et. al., QEEG and neurofeedback for assessment and effective intervention with attention deficit hyperactivity disorder, In Introduction to Quantitative EEG and Neurofeedback: Advanced Theory and Applications, pages 337-364, Elsevier, 2 edition, 2009.

There are typically two major neurofeedback approaches, namely, self-regulation of band powers and/or their ratios, and of slow-cortical potentials. However, the neurofeedback techniques above are typically based on extrapolations from research describing abnormal EEG patterns in subjects with ADHD. Such a generic model approach may not apply well to each and every patient. For example, in M. Thompson et. al., Improving attention in adults and children: Differing electroencephalography profiles and implications for training, Biofeedback, 34:99-105, 2006, it was discussed that regardless of age, each client/patient is unique. Thus, the intervention should be customized.

A treatment method beyond the neurofeedback approach has been proposed. In the treatment, individual subjects' attention conditions, decoded from EEG signals using a subject-specific attention detection model are associated directly with a control signal in therapeutic video games. In a pilot trial involving 10 intervention subjects versus 10 controls, significant improvement in ADHD rating scores were observed especially in inattentive symptoms.

However, it has been recognised that there are two main problems associated with the above attention detection model/algorithm. Firstly, the attention detection model is sensitive to eye movement artefacts. That is, if a user blinks his/her eyes in a concentration state, the algorithm produces false negatives. Thus, the user must minimize eye blinks during playing of attention-driven video games, which can typically be frustrating, especially during relatively long time sessions. Secondly, it has been found that the classification accuracy of the algorithm is relatively low for some subjects, such that the specificity of attention detection outcomes and the treatment effects may be significantly limited.

Hence, in view of the above, there exists a need for a method and system for detecting attention that seek to address at least one of the above problems.

### SUMMARY

In accordance with an aspect of the present invention, there is provided a method of detecting attention of a subject, the method comprising determining one or more user specific feature sets from Electroencephalographic (EEG) signals of the subject; determining one or more pool data feature sets from the EEG signals of the subject; identifying one or more features from each feature set for differentiating attention/non-attention signals; determining respective classification scores for each feature set based on the identified one or more features; and combining the classification scores to obtain a combined attention score for said detecting attention.

A first one of the user specific feature sets may be based on each feature of the feature set being an average band-power over a time-window of a differential potential between two EEG channels coupled to the subject and a second one of the user specific feature sets may be based on each feature of the feature set being an average band-power over a time-window of a differential potential between two EEG channels coupled to the subject after normalization to uni-variance.

A first one of the pool data feature sets may be based on each feature of the feature set being an average band-power over a time-window of a differential potential between two EEG channels coupled to the subject as compared to EEG data of a pool of subjects and a second one of the pool data feature sets may be based on each feature of the feature set being an average band-power over a time-window of a differential potential between two EEG channels coupled to the subject as compared to said EEG data of the pool of subjects after normalization to uni-variance.

The step of identifying one or more features from each feature set may comprise maximising mutual information I between brain conditions C (either attention or in-attentiveness) and EEG features A from the features sets using:

$$I ( A , C ) = H ( C ) - H ( C \vert A ) = {\sum }_{ω} {\int }_{a} {p}_{a , ω} ( a , ω ) log ⌊ \frac{{ρ}_{a , ω} ( a , ω )}{{ρ}_{a} ( a ) {P}_{ω} ( ω )} ⌋ d a$$

where H(C) is an attention condition variable ω, and H(C|A) is a conditional entropy of an attention condition if the features are known, and a is a feature vector, and P (or p) denotes a probability (or probability density) function.

The step of combining the classification scores may comprise using a Fishier linear discriminant (FLD) to combine the classification scores.

In accordance with another aspect of the present invention, there is provided a system for attention detection of a subject, the system comprising a processing module, the processing module capable of determination of one or more user specific feature sets from Electroencephalographic (EEG) signals of the subject; determination of one or more pool data feature sets from the EEG signals of the subject; identification of one or more features from each feature set for differentiating attention/non-attention signals; determination of respective classification scores for each feature set based on identified one or more features; and combination of the classification scores to obtain a combined attention score for said attention detection.

A first one of the user specific feature sets may be based on each feature of the feature set being an average band-power over a time-window of a differential potential between two EEG channels coupled to the subject and a second one of the user specific feature sets may be based on each feature of the feature set being an average band-power over a time-window of a differential potential between two EEG channels coupled to the subject after normalization to uni-variance.

A first one of the pool data feature sets may be based on each feature of the feature set being an average band-power over a time-window of a differential potential between two EEG channels coupled to the subject as compared to EEG data of a pool of subjects and a second one of the pool data feature sets may be based on each feature of the feature set being an average band-power over a time-window of a differential potential between two EEG channels coupled to the subject as compared to said EEG data of the pool of subjects after normalization to uni-variance.

The processing module may identify one or more features from each feature set by maximisation of mutual information I between brain conditions C (either attention or in-attentiveness) and EEG features A from the features sets by utilisation of:

$$I ( A , C ) = H ( C ) - H ( C \vert A ) = {\sum }_{ω} {\int }_{a} {p}_{a , ω} ( a , ω ) log ⌊ \frac{{ρ}_{a , ω} ( a , ω )}{{ρ}_{a} ( a ) {P}_{ω} ( ω )} ⌋ d a$$

where H(C) is an attention condition variable ω, and H(C|A) is a conditional entropy of an attention condition if the features are known, and a represents a feature vector, and P (or p) denotes a probability (or probability density) function.

The processing module may combine the classification scores by utilisation of a Fishier linear discriminant (FLD).

In accordance with another aspect of the present invention, there is provided a computer readable data storage medium having stored thereon computer code means for instructing a computer processor to execute a method of detecting attention of a subject, the method comprising determining one or more user specific feature sets from Electroencephalographic (EEG) signals of the subject; determining one or more pool data feature sets from the EEG signals of the subject; identifying one or more features from each feature set for differentiating attention/non-attention signals; determining respective classification scores for each feature set based on the identified one or more features; and combining the classification scores to obtain a combined attention score for said detecting attention.

In accordance with yet another aspect of the present invention, there is provided a method of detecting attention of a subject, the method comprising determining a plurality of feature sets from Electroencephalographic (EEG) signals of the subject; identifying one or more features from each feature set for differentiating attention/non-attention signals; determining respective classification scores for each feature set based on the identified one or more features; combining the classification scores to obtain a combined attention score for said detecting attention; and wherein a first one of the feature sets is based on each feature of the feature set being an average band-power over a time-window of a differential potential between two EEG channels coupled to the subject and a second one of the feature sets is based on each feature of the feature set being an average band-power over a time-window of a differential potential between two EEG channels coupled to the subject after normalization to uni-variance.

The feature sets may comprise user specific feature sets associated to the subject.

The feature sets may comprise pool data feature sets based on EEG data from a pool of subjects.

The step of identifying one or more features from each feature set may comprise maximising mutual information I between brain conditions C (either attention or in-attentiveness) and EEG features A from the features sets using:

$$I ( A , C ) = H ( C ) - H ( C \vert A ) = {\sum }_{ω} {\int }_{a} {p}_{a , ω} ( a , ω ) log ⌊ \frac{{ρ}_{a , ω} ( a , ω )}{{ρ}_{a} ( a ) {P}_{ω} ( ω )} ⌋ d a$$

where H(C) is an attention condition variable ω, and H(C|A) is a conditional entropy of an attention condition if the features are known, and a is a feature vector, and P (or p) denotes a probability (or probability density) function.

The step of combining the classification scores may comprise using a Fishier linear discriminant (FLD) to combine the classification scores.

In accordance with another aspect of the present invention, there is provided a system for attention detection of a subject, the system comprising a processing module, the processing module capable of determination of a plurality of feature sets from Electroencephalographic (EEG) signals of the subject; identification of one or more features from each feature set for differentiating attention/non-attention signals; determination of respective classification scores for each feature set based on identified one or more features; and combination of the classification scores to obtain a combined attention score for said attention detection; and wherein a first one of the feature sets is based on each feature of the feature set being an average band-power over a time-window of a differential potential between two EEG channels coupled to the subject and a second one of the feature sets is based on each feature of the feature set being an average band-power over a time-window of a differential potential between two EEG channels coupled to the subject after normalization to uni-variance.

The feature sets may comprise user specific feature sets associated to the subject.

The feature sets may comprise pool data feature sets based on EEG data from a pool of subjects.

The processing module may identify one or more features from each feature set by maximisation of mutual information I between brain conditions C (either attention or in-attentiveness) and EEG features A from the features sets by utilisation of:

$$I ( A , C ) = H ( C ) - H ( C \vert A ) = {\sum }_{ω} {\int }_{a} {p}_{a , ω} ( a , ω ) log ⌊ \frac{{ρ}_{a , ω} ( a , ω )}{{ρ}_{a} ( a ) {P}_{ω} ( ω )} ⌋ d a$$

where H(C) is an attention condition variable ω, and H(C|A) is a conditional entropy of an attention condition if the features are known, and a is a feature vector, and P (or p) denotes a probability (or probability density) function.

The processing module may combine the classification scores by utilisation of a Fishier linear discriminant (FLD).

In accordance with another aspect of the present invention, there is provided a computer readable data storage medium having stored thereon computer code means for instructing a computer processor to execute a method of detecting attention of a subject, the method comprising determining a plurality of feature sets from Electroencephalographic (EEG) signals of the subject; identifying one or more features from each feature set for differentiating attention/non-attention signals; determining respective classification scores for each feature set based on the identified one or more features; combining the classification scores to obtain a combined attention score for said detecting attention; and wherein a first one of the feature sets is based on each feature of the feature set being an average band-power over a time-window of a differential potential between two EEG channels coupled to the subject and a second one of the feature sets is based on each feature of the feature set being an average band-power over a time-window of a differential potential between two EEG channels coupled to the subject after normalization to uni-variance.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1(a) is a schematic diagram illustrating an attention training system in an example embodiment.

FIG. 1(b) is an enlarged view of a processing module of FIG. 1(a).

FIG. 2 is a graph illustrating comparative detection accuracy in User Test 1 in an example embodiment.

FIG. 3 is a graph illustrating comparative detection accuracy in. User Test 2 in an example embodiment.

FIG. 4 is a schematic flowchart illustrating a method of detecting attention of a subject in an example embodiment.

FIG. 5 is a schematic diagram of a computer system for implementing a method and system of detecting attention of a subject in an example embodiment.

### DETAILED DESCRIPTION

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “scanning”, “calculating”, “determining”, “replacing”, “generating”, “initializing”, “outputting”, or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a general purpose computer or other device selectively activated or reconfigured by a computer program stored in the computer. The algorithms and displays presented herein are not inherently related to any particular computer or other apparatus. Various general purpose machines may be used with programs in accordance with the teachings herein. Alternatively, the construction of more specialized apparatus to perform the required method steps may be appropriate. The structure of a conventional general purpose computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a general purpose computer. The computer readable medium may also include a hard-wired medium such as exemplified in the Internet system, or wireless medium such as exemplified in the GSM mobile telephone system. The computer program when loaded and executed on such a general-purpose computer effectively results in an apparatus that implements the steps of the preferred method.

The invention may also be implemented as hardware modules. More particular, in the hardware sense, a module is a functional hardware unit designed for use with other components or modules. For example, a module may be implemented using discrete electronic components, or it can form a portion of an entire electronic circuit such as an Application Specific integrated Circuit (ASIC). Numerous other possibilities exist. Those skilled in the art will appreciate that the system can also be implemented as a combination of hardware and software modules.

In an example embodiment, an algorithm comprising computational models of attention detection can address various artefacts, including those from eye-blinking and low quality EEG recordings.

In the example embodiment, continuous EEG signals are decoded into a time sequence of attention scores that represent a user's conditions of attention at each respective time point.

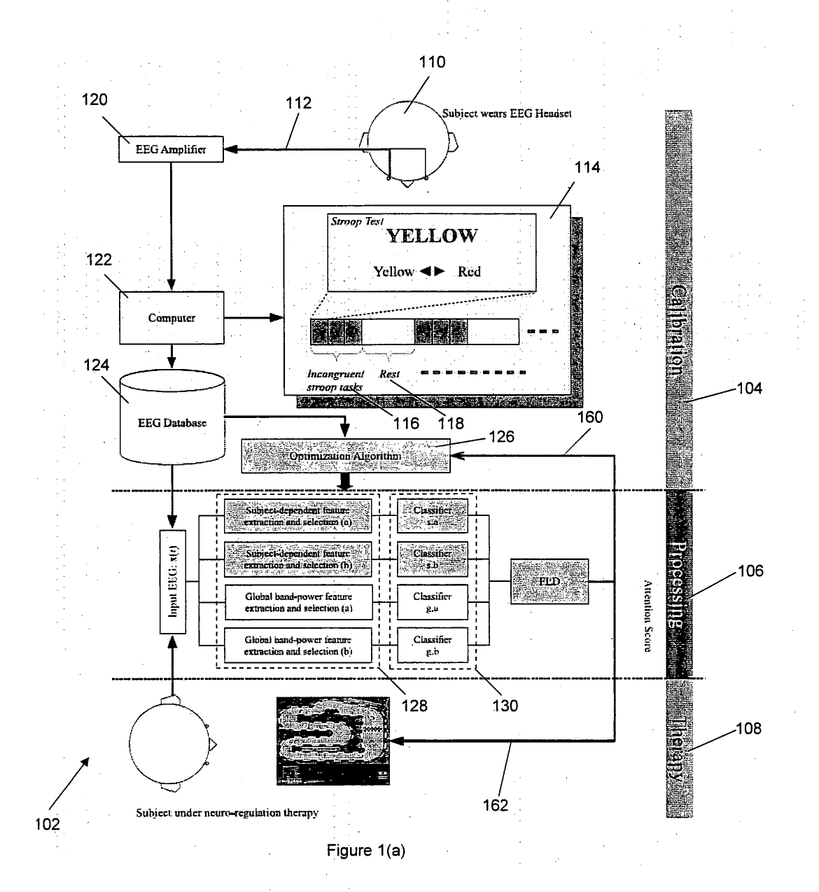

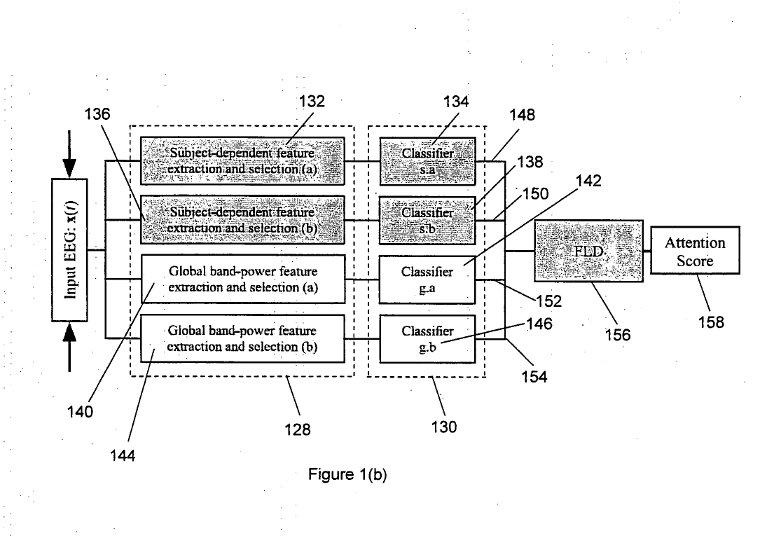

FIG. 1(a) is a schematic diagram illustrating an attention training system in an example embodiment. The system 102 can learn parameters and can be applied in a video game. The system 102 comprises a calibration module 104, a processing module 106 and a therapy module 108.

In the calibration module 104, a subject/patient 110 provides EEG signals 112 while performing an instructed task 114. The task 114 comprises interleaved incongruent stroop tasks 116 and resting tasks 118. The concurrent EEG signals 112 during performance of the task 114 are recorded using, for example, a device from Neuroscan. The device can comprise an EEG amplifier 120 coupled to a computer 122. The computer 122 administers the task 114 and maintains en EEG database 124.

In the example embodiment, the sampling rate of the device, which refers to the speed of an analog-digital converter of the device, is preferably set to no less than 100 Hz, more preferably about 250 Hz. The sampling rate is chosen to contain useful EEG rhythms. Two EEG channels of the device are used to measure electrical potentials at pre-frontal locations of the subject 110. In the example embodiment, the two EEG channels are connected to the left and right sides of the subject's 110 forehead. It has been recognised that the frontal lobe of a brain and probably the parietal part contribute to attention activities. This is discussed in J. V. Pardo et. al., The anterior cingulate cortex mediates processing selection in the stroop attentional conflict paradigm, PNAS, 87:256-259, 1990, which is incorporated herein by reference.

In the example embodiment, the incongruent Stroop tasks 116 are employed because they preferably correspond to the executive attention network of the brain, which may be related to inattentiveness in ADHD patients. This is discussed in M. M. Lansbergen et. al., Stroop interference and attention-deficit/hyperactivity disorder: a review and meta-analysis, Neuropsychology, 21:251262, 2007, which is incorporated herein by reference. On the other hand, for the resting tasks 118, the subject 110 is instructed not to pay attention to the game/task 114 or to anything else. Therefore, the resting tasks 118 are expected to generate more likely inattentiveness than in the Stroop tasks 116.

In the example embodiment, the processing module 106 implements an optimisation algorithm 126. The optimisation or attention detection algorithm 126 comprises feature selection 128 and classification 130. It has been recognised that selection of spatial-spectrum features may be a challenge in attention. detection. In the example embodiment, two sets of features are considered.

In the first set, set (a), each feature is the average band-power over a time-window of the differential potential between two EEG channels. In the second set, set (b), each feature is the average band-power over a time-window of the differential potential between two EEG channels after normalization to uni-variance. The two sets (a) and (b) are chosen as such to capture the rhythmic power of each EEG channel, or that of the differential potential between the channels. It has been recognised that rhythmic powers are a source of information for determining brain activities from EEG.

To identify significant features for differentiating the attention/non-attention brain signals, mutual information I between the brain conditions C (either attention or in-attentiveness) and the selected EEG features A (from set (a) or (b)) is maximised as follows:

$$I ( A , C ) = H ( C ) - H ( C \vert A ) = {\sum }_{ω} {\int }_{a} {p}_{a , ω} ( a , ω ) log ⌊ \frac{{ρ}_{a , ω} ( a , ω )}{{ρ}_{a} ( a ) {P}_{ω} ( ω )} ⌋ d a \qquad ( 1 )$$

where H(C) is the attention condition variable ω, and H(C|A)the conditional entropy of the attention condition if the features are known. Here a is a particular feature vector, and P (or p) denotes the probability (or probability density) function. Entropy is related to the uncertainty of a random variable. Hence, the mutual information I(A,C) is the reduction of uncertainty about attention conditions by knowing the EEG features. This is discussed in T. M. Cover et. al., Elements of Information Theory, Wiley, New York, 2nd edition, 2006, which is incorporated herein by reference. The mutual information can be estimated using a Monte-Carlo method described in H. Zhang et. al., Spatio-spectral feature selection based on robust mutual information estimate for brain computer interfaces, In Annual International Conference of the IEEE Engineering in Medicine and Biology Society, pages 2391-2398, 2009, which is incorporated herein by reference. Hence, optimum spectral filters can be selected from a number of pre-defined band-pass filters such that the mutual information is empirically maximized.

In the example embodiment, the identification of the features is separate in the two feature sets, i.e. sets (a) and (b). A linear classifier (e.g. a Fishier linear discriminant (FLD)) (compare 134, 138, 142, 146 of FIG. 1(b)) is used to map each feature set into a score. As attention is considered a positive class and inattention is considered a negative class, a higher score means a higher level of attention.

FIG. 1(b) is an enlarged view of the processing module 106 of FIG. 1(a). For set (a), there is provided a subject-dependent feature extraction and selection module (a) 132 and a respective classifier (s.a) 134, as a pair. For set (b), there is provided a subject-dependent feature extraction and selection module (b) 136 and a respective classifier (s.b) 138, as a pair. In addition to the two pairs of feature set/module 132, 136 and respective classifiers 134, 138, another two pairs of feature set/module and classifier are also considered. These are a global band-power feature extraction and selection module (a) 140 and a respective classifier (g.a) 142 and a global band-power feature extraction and selection module (b) 144 and a respective classifier (g.b) 146. The two pairs of feature set/module 140, 144 and classifier 142, 146 are learnt from a pool of users to allow exploration of common features in brain signals across different subjects/users so as to improve the robustness of the system 102.

In the example embodiment, each pair of feature set/module and classifier generates a single score. The four scores 148, 150, 152, 154 are combined into a joint score vector. A Fishier linear discriminant (FLD) 156 is provided to take the joint score vector as input and to provide a final attention score 158 as output. The final attention score 158 may be used for therapy purposes or as a means of game control (see numeral 162) in the therapy module 108 and/or for tuning the optimisation algorithm (see numeral 160).

In the above described example embodiment, the algorithm can address the two issues of eye blinking artefacts and low classification. It has also been found that the set (b) pairs, i.e. numerals 136-138 pair and numerals 144-146 pair, are generally more robust against blinking artefacts than the other two pairs for set (a).

In an example embodiment, a training/attention detection algorithm is provided. As input to the algorithm, n samples of EEG segments extracted from attention and non-attention conditions are provided. The algorithm provides as output subject-specific feature extraction modules (s.a and s.b) (compare 132, 136 of FIG. 1(b)) together with respective classifiers (s.a and s.b) (compare 134, 138 of FIG. 1(b), and a classifier (compare 156 of FIG. 1(b)) that combines the two subject-specific scores (compare 148, 150 of FIG. 1(b)) together with scores generated by global models (compare 152, 154 of FIG. 1(b)).

In the example embodiment, as a first step, the algorithm learns/determines a subject-specific feature extraction module (s.a). This is achieved by computing the average band powers in the differential potential between the two EEG channels over a time-window. In the example embodiment, seven 4 Hz-wide frequency bands whose start points are evenly distributed in the range from about 4 Hz to about 28 Hz are computed. A greedy iterative search procedure is then applied to identify the significant features that together produce the largest mutual information. Next, the algorithm learns a linear classifier that maps the selected/identified features to class labels.

As a second step, the algorithm learns/determines another subject-specific feature extraction module (s.b). The second step is substantially identical to the first step except the algorithm normalizes each EEG channel to uni-variance in the time-window before computing the band power features.

As a third step, the algorithm forms a joint score vector from the two classifier outputs (compare 148, 150 of FIG. 1(b) in addition to two “global models” outputs (compare 152, 154 of FIG. 1(b)). In the example embodiment, the global models are built in advance with substantially the same techniques in Step 1 and Step 2 but using an aggregated data pool from a plurality of subjects.

As a fourth step, the algorithm learns the linear classification that maps the joint score vector to the class labels. The continuous output of the classifier (compare 156 of FIG. 1(b)) is taken as the final attention score (compare 158 of FIG. 1(b)). The classifier used in the example embodiment can include any classifier, for example, a FLD.

In an example implementation of the algorithm, 18 subjects were recruited into a user test study. Each subject performed a Stroop test for collection of concentration/non-concentration EEG signals. In the example, the Stroop test comprises 20 pairs of Stroop task blocks and relaxation blocks. In each Stroop task block of about 6 seconds in duration, the subject responded to a question comprising approximately 6 seconds of Stroop test. Each relaxation block matches in duration to the preceding Stroop task block.

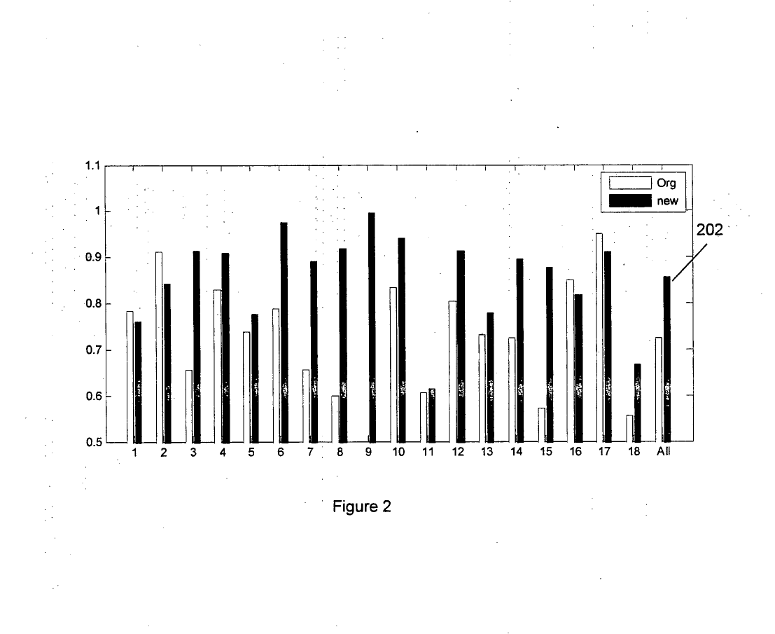

All the 18 subjects participated in one round of user test. This first round of user test is denoted as User Test 1. FIG. 2 is a graph illustrating the comparative detection accuracy in User. Test 1. The horizontal axis denotes the human subject number, for example, from 1 to 18 in User Test 1. The “All” item refers to the average result over all the subjects in User Test 1. The vertical axis denotes the detection accuracy rate, i.e. the number of EEG time windows being misclassified versus the number of all the EEG time windows. The “Org” bars denote the results generated by a prior-art method published under WO/2009/134205, while the “new” bars show those by the algorithm of the example embodiment.

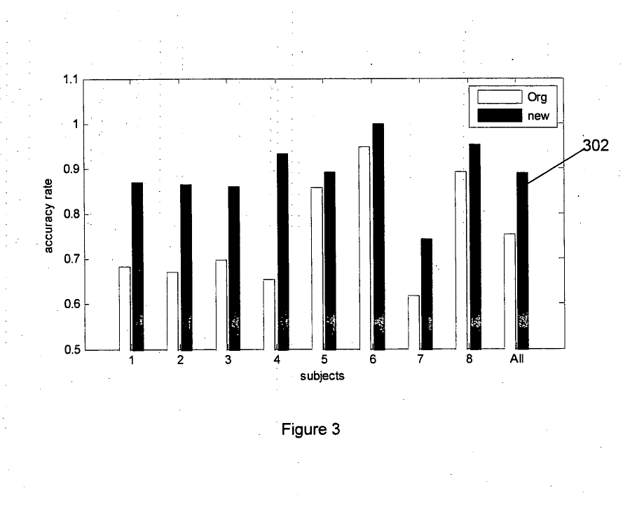

In addition, eight subjects participated in an additional round of user test. This additional round of user test is denoted as User Test 2. FIG. 3 is a graph illustrating the comparative detection accuracy in User Test 2.

In the example implementation, overall, the statistics show that the subjects achieved a high accuracy of 0.99±0.01 (in mean and standard deviation) in Stroop test responses. The response time was about 2.72±0.10 seconds (not shown). The user performances indicate a satisfactory quality of the data. The proposed algorithm is observed to deliver overall the highest detection accuracy in both user test cases (see numerals 202 and 302). It can significantly improve the accuracy from a level of about 70% to a level of more than about 85% (see numerals 202 and 302).

Furthermore, the continuous output of the algorithm was examined. It was found that the algorithm is more robust against eye-blinking artefacts as compared to the prior-art method and correctly produced positive scores during the concentration period.

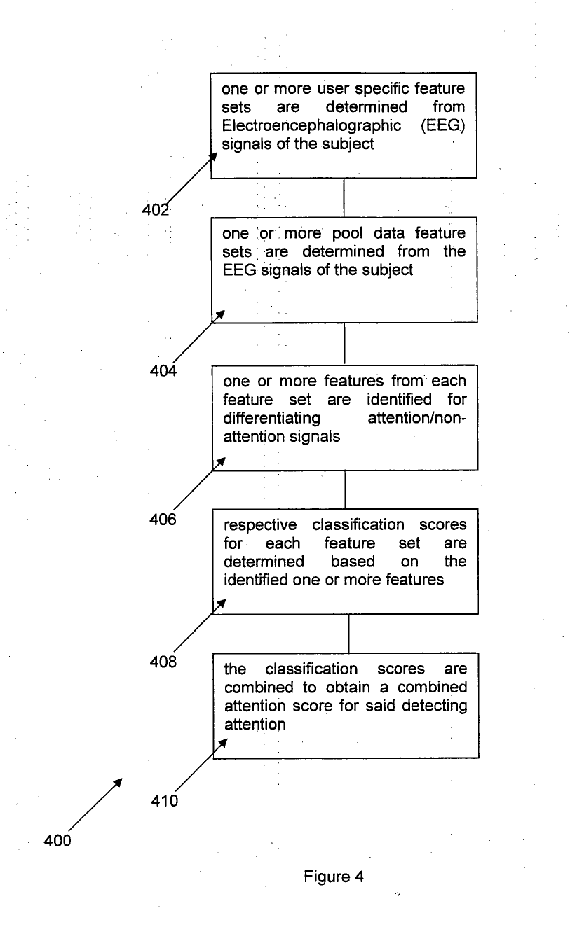

FIG. 4 is a schematic flowchart 400 illustrating a method of detecting attention of a subject in an example embodiment. At step 402, one or more user specific feature sets are determined from Electroencephalographic (EEG) signals of the subject. At step 404, one or more pool data feature sets are determined from the EEG signals of the subject. At step 406, one or more features from each feature set are identified for differentiating attention/non-attention signals. At step 408, respective classification scores for each feature set are determined based on the identified one or more features. At step 410, the classification scores are combined to obtain a combined attention score for said detecting attention.

In the above described example embodiments, a new algorithm can be provided that significantly improves attention detection performance. Detection output can be made insensitive to eye blinks and the algorithm can be used for attention training to obtain significantly higher specificity of attention training and higher usability.

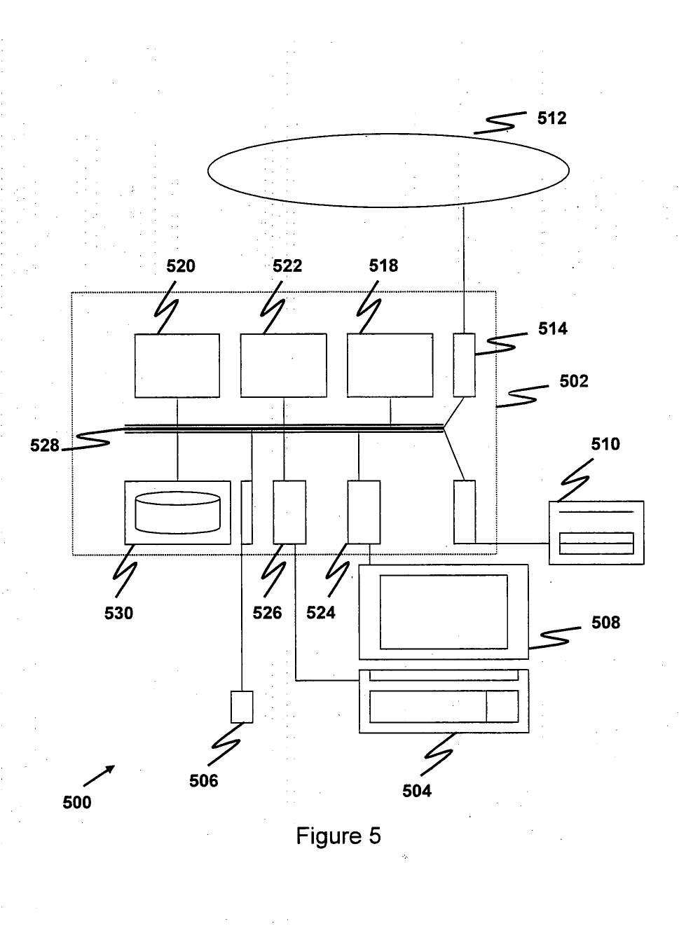

The method and system of the example embodiments can be implemented on a computer system 500, schematically shown in FIG. 5. It may be implemented as software, such as a computer program being executed within the computer system 500, and instructing the computer system 500 to conduct the method of the example embodiment.

The computer system 500 comprises a computer module 502, input modules such as a keyboard 504 and mouse 506 and a plurality of output devices such as a display 508, and printer 510.

The computer module 502 is connected to a computer network 512 via a suitable transceiver device 514, to enable access to e.g. the Internet or other network systems such as Local Area Network (LAN) or Wide Area Network (WAN).

The computer module 502 in the example includes a processor 518, a Random Access Memory (RAM) 520 and a Read Only Memory (ROM) 522. The computer module 502 also includes a number of Input/Output (I/O) interfaces, for example I/O interface 524 to the display 508, and I/O interface 526 to the keyboard 504.

The components of the computer module 502 typically communicate via an interconnected bus 528 and in a manner known to the person skilled in the relevant art. The application program is typically supplied to the user of the computer system 500 encoded on a data storage medium such as a CD-ROM or flash memory carrier and read utilising a corresponding data storage medium drive of a data storage device 530. The application program is read and controlled in its execution by the processor 518. Intermediate storage of program data maybe accomplished using RAM 520.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1. A method of detecting attention of a subject, the method comprising, determining one or more user specific feature sets from Electroencephalographic (EEG) signals of the subject;
determining one or more pool data feature sets from the EEG signals of the subject;
identifying one or more features from each feature set for differentiating attention/non-attention signals;
determining respective classification scores for each feature set based on the identified one or more features; and
combining the classification scores to obtain a combined attention score for said detecting attention.

2. The method as claimed in claim 1, wherein a first one of the user specific feature sets is based on each feature of the feature set being an average band-power over a time-window of a differential potential between two EEG channels coupled to the subject and a second one of the user specific feature sets is based on each feature of the feature set being an average band-power over a time-window of a differential potential between two EEG channels coupled to the subject after normalization to uni-variance.

3. The method as claimed in claim 1, wherein a first one of the pool data feature sets is based on each feature of the feature set being an average band-power over a time-window of a differential potential between two EEG channels coupled to the subject as compared to EEG data of a pool of subjects and a second one of the pool data feature sets is based on each feature of the feature set being an average band-power over a time-window of a differential potential between two EEG channels coupled to the subject as compared to said EEG data of the pool of subjects after normalization to uni-variance.

4. The method as claimed in claim 1, wherein the step of identifying one or more features from each feature set comprises maximising mutual information I between brain conditions C (either attention or in-attentiveness) and EEG features A from the features sets using:
$$I ( A , C ) = H ( C ) - H ( C \vert A ) = {\sum }_{ω} {\int }_{a} {p}_{a , ω} ( a , ω ) log ⌊ \frac{{ρ}_{a , ω} ( a , ω )}{{ρ}_{a} ( a ) {P}_{ω} ( ω )} ⌋ d a$$
where H(C) is an attention condition variable ω, and H(C|A) is a conditional entropy of an attention condition if the features are known, and a is a particular feature vector, and P (or p) denotes a probability (or probability density) function.

5. The method as claimed in claim 1, wherein the step of combining the classification scores comprises using a Fishier linear discriminant (FLD) to combine the classification scores.

6. A system for attention detection of a subject, the system comprising
a processing module, the processing module capable of determination of one or more user specific feature sets from Electroencephalographic (EEG) signals of the subject;
determination of one or more pool data feature sets from the EEG signals of the subject;
identification of one or more features from each feature set for differentiating attention/non-attention signals;
determination of respective classification scores for each feature set based on identified one or more features; and
combination of the classification scores to obtain a combined attention score for said attention detection.

7. The system as claimed in claim 6, wherein a first one of the user specific feature sets is based on each feature of the feature set being an average band-power over a time-window of a differential potential between two EEG channels coupled to the subject and a second one of the user specific feature sets is based on each feature of the feature set being an average band-power over a time-window of a differential potential between two EEG channels coupled to the subject after normalization to uni-variance.

8. The system as claimed in claim 6, wherein a first one of the pool data feature sets is based on each feature of the feature set being an average band-power over a time-window of a differential potential between two EEG channels coupled to the subject as compared to EEG data of a pool of subjects and a second one of the pool data feature sets is based on each feature of the feature set being an average band-power over a time-window of a differential potential between two EEG channels coupled to the subject as compared to said EEG data of the pool of subjects after normalization to uni-variance.

9. The system as claimed in claim 6, wherein the processing module identifies one or more features from each feature set by maximisation of mutual information I between brain conditions C (either attention or in-attentiveness) and EEG features A from the features sets by utilisation of:
$$I ( A , C ) = H ( C ) - H ( C \vert A ) = {\sum }_{ω} {\int }_{a} {p}_{a , ω} ( a , ω ) log ⌊ \frac{{ρ}_{a , ω} ( a , ω )}{{ρ}_{a} ( a ) {P}_{ω} ( ω )} ⌋ d a$$
where H(C) is an attention condition variable ω, and H(C|A) is a conditional entropy of an attention condition if the features are known, and a is a particular feature vector, and P (or p) denotes a probability (or probability density) function.

10. The system as claimed in claim 6, wherein the processing module combines the classification scores by utilisation of a Fishier linear discriminant (FLD).

11. A computer readable data storage medium having stored thereon computer code means for instructing a computer processor to execute a method of detecting attention of a subject, the method comprising,
determining one or more user specific feature sets from Electroencephalographic (EEG) signals of the subject;
determining one or more pool data feature sets from the EEG signals of the subject;
identifying one or more features from each feature set for differentiating attention/non-attention signals;
determining respective classification scores for each feature set based on the identified one or more features; and
combining the classification scores to obtain a combined attention score for said detecting attention.

12. A method of detecting attention of a subject, the method comprising, determining a plurality of feature sets from Electroencephalographic (EEG) signals of the subject;
identifying one or more features from each feature set for differentiating attention/non-attention signals;
determining respective classification scores for each feature set based on the identified one or more features;
combining the classification scores to obtain a combined attention score for said detecting attention; and
wherein a first one of the feature sets is based on each feature of the feature set being an average band-power over a time-window of a differential potential between two EEG channels coupled to the subject and a second one of the feature sets is based on each feature of the feature set being an average band-power over a time-window of a differential potential between two EEG channels coupled to the subject after normalization to uni-variance.

13. The method as claimed in claim 12, wherein the feature sets comprise user specific feature sets associated to the subject.

14. The method as claimed in claim 12, wherein the feature sets comprise pool data feature sets based on EEG data from a pool of subjects.

15. The method as claimed in claim 12, wherein the step of identifying one or more features from each feature set comprises maximising mutual information I between brain conditions C (either attention or in-attentiveness) and EEG features A from the features sets using:
$$I ( A , C ) = H ( C ) - H ( C \vert A ) = {\sum }_{ω} {\int }_{a} {p}_{a , ω} ( a , ω ) log ⌊ \frac{{ρ}_{a , ω} ( a , ω )}{{ρ}_{a} ( a ) {P}_{ω} ( ω )} ⌋ d a$$
where H(C) is an attention condition variable ω, and H(C|A) is a conditional entropy of an attention condition if the features are known, and a is a particular feature vector, and P (or p) denotes a probability (or probability density) function.

16. The method as claimed in claim 12, wherein the step of combining the classification scores comprises using a Fishier linear discriminant (FLD) to combine the classification scores.

17. A system for attention detection of a subject, the system comprising
a processing module, the processing module capable of
determination of a plurality of feature sets from Electroencephalographic (EEG) signals of the subject;
identification of one or more features from each feature set for differentiating attention/non-attention signals;
determination of respective classification scores for each feature set based on identified one or more features; and
combination of the classification scores to obtain a combined attention score for said attention detection; and
wherein a first one of the feature sets is based on each feature of the feature set being an average band-power over a time-window of a differential potential between two EEG channels coupled to the subject and a second one of the feature sets is based on each feature of the feature set being an average band-power over a time-window of a differential potential between two EEG channels coupled to the subject after normalization to uni-variance.

18. The system as claimed in claim 17, wherein the feature sets comprise user specific feature sets associated to the subject.

19. The system as claimed in claim 17, wherein the feature sets comprise pool data feature sets based on EEG data from a pool of subjects.

20. The system as claimed in claim 17, wherein the processing module identifies one or more features from each feature set by maximisation of mutual information I between brain conditions C (either attention or in-attentiveness) and EEG features A from the features sets by utilisation of:
$$I ( A , C ) = H ( C ) - H ( C \vert A ) = {\sum }_{ω} {\int }_{a} {p}_{a , ω} ( a , ω ) log ⌊ \frac{{ρ}_{a , ω} ( a , ω )}{{ρ}_{a} ( a ) {P}_{ω} ( ω )} ⌋ d a$$
where H(C) is an attention condition variable ω, and H(C|A) is a conditional entropy of an attention condition if the features are known, and a is a particular feature vector, and P (or p) denotes a probability (or probability density) function.

21. The system as claimed in claim 17, wherein the processing module combines the classification scores by utilisation of a Fishier linear discriminant (FLD).

22. A computer readable data storage medium having stored thereon computer code means for instructing a computer processor to execute a method of detecting attention of a subject, the method comprising,
determining a plurality of feature sets from Electroencephalographic (EEG) signals of the subject;
identifying one or more features from each feature set for differentiating attention/non-attention signals;
determining respective classification scores for each feature set based on the identified one or more features;
combining the classification scores to obtain a combined attention score for said detecting attention; and
wherein a first one of the feature sets is based on each feature of the feature set being an average band-power over a time-window of a differential potential between two EEG channels coupled to the subject and a second one of the feature sets is based on each feature of the feature set being an average band-power over a time-window of a differential potential between two EEG channels coupled to the subject after normalization to uni-variance.
